(19) United States
(12) Patent Application Publication
(10) Pub. No.: US 2008/0109170 A1
(43) Pub. Date: May 8, 2008

(54) Method of presentation of data of stored body energy

(75) Inventor: Russ E. Lindemann, Valders, WI (US)

Correspondence Address: Russ E. Lindemann, PO Box 340, Maintowoc, WI, 54221-0340, US

(73) Assignee: Russ E. Lindemann, Valders, WI (US)

(21) Appl. No.: 11/593,877

(22) Filed: Nov. 6, 2006

(51) Int. Cl. G06F 19/00; G01N 33/48

(52) U.S. Cl. 702/19

(57) ABSTRACT

Disclosed is a method of calculating energy stored in a body and creating a data set of information and using that information to guide the owner of a body to achieve a desired stored energy quantity.

### CROSS-REFERENCE TO RELATED APPLICATIONS

Not Applicable

### STATEMENT REGARDING FEDERALLY SPONSERED RESEARCH OR DEVELOPMENT (IF ANY)

Not Applicable

### REFERENCE TO SEQUENCE LISTING, A TABLE, OR A COMPUTER PROGRAM LISTING COMPACT DISC APPENDIX

Not Applicable

### BACKGROUND OF INVENTION

1. Field of Endeavor

This invention relates to any method, system, or procedure to calculate stored body energy.

Energy stored as body fat, especially in humans, can be extremely unhealthy if it becomes disproportionately balanced. It is in the best interest of the health of a body, whether human or animal, to keep stored body energy within a given tolerance for a particular size body. There is much misinformation available on this subject to the general public. The energy needs and storage ability can be projected within a reasonable amount of accuracy, thus allowing the ability with a high amount of accuracy to project energy gain or loss of a body's energy storage reserves.

It has long been known that accounting for energy consumed is the best means of monitoring projected stored energy levels. The ability to project stored energy level allows the opportunity to uniquely account for energy stored by means of accessing a body's current stored energy level and adding or subtracting the surplus or deficit difference of the daily energy needs of a body against the actual daily energy consumed by the body.

These figures coupled with a body's gender and size afford the ability to create a wide variety of data for an individual body which can be presented by means of visual, audio, or other such representation.

### BRIEF SUMMARY OF INVENTION

A method of calculating energy stored in a body and creating a data set of information and using that information to guide the owner of a body to achieve a desired stored energy quantity. So that the body owner will have a reasonably accurate projection of what their stored body energy quantity should be at a given point in time.

### BRIEF DESCRIPTION OF THE SEVERAL VIEWS OF THE DRAWING

Not Applicable

### DETAILED DESCRIPTION OF THE INVENTION

A method of calculating energy stored in a body and creating a data set of information and using that information to guide the owner of a body to achieve a desired stored energy quantity has many potential applications. A prominent application being, based on the pertinent characteristics of a body, to estimate stored energy (fat) in a body and estimate the energy needs for a body for a given period of time (such as a day) and monitoring of the energy intake for said time period then determining the surplus or deficit of energy consumed by determining the difference of the estimated energy needs for a given body for a given time period and the energy intake for said time period then adding or subtracting that difference to or from the estimated stored energy of the body for each time period in the created data set thus allowing the owner of the body the ability to understand the reasonable estimate of projected stored energy for any point in time.

A textual illustration would be if a body had 80,000 excess calories of energy stored as fat and had a daily need of 2,000 calories but consumed only 1,000 calories per day, that body would be consuming a 1,000 calorie deficit which would be subtracted from the 80,000 excess calories leaving 79,000 calories left. If this body were to be on a regular daily schedule, doing this for an additional 79 days, it would then take 80 days total at that particular rate to consume (as energy used) all 80,000 excess calories of energy. Upon presentation of this data set schedule, it would be known to the owner of the body that the excess energy stored will be consumed by the body in approximately 80 days total, thus presenting a clear and concise time period goal for the owner of the body to achieve desired energy storage quantity (body weight).

Along with many other potential uses of this method, the inverse could also be used to increase the stored energy level of a body by exceeding the estimated daily energy needs of a body and accumulating that until the desired stored energy level is achieved.

1. A method of projecting and presenting data for energy stored within a body for any given period of time and accumulating and presenting that data as a running total for each time period in a data set.
